(19) United States
(12) Patent Application Publication
(10) Pub. No.: US 2025/0318769 A1
(43) Pub. Date: Oct. 16, 2025

(54) Evaluation method of the fatigue level of an operator and associated evaluation system

(71) Applicant: THALES, Meudon (FR)

(72) Inventors: Florence DURIEUX, Merignac (FR); Lionel GRANIER, Merignac (FR); Vincent IBANEZ, Merignac (FR)

(21) Appl. No.: 19/172,644

(22) Filed: Apr. 7, 2025

(30) Foreign Application Priority Data
Apr. 12, 2024 (FR) 2403800

(51) Int. Cl. A61B 5/16; A61B 5/00; A61B 5/18; G16H 10/20; G16H 40/67

(52) U.S. Cl. CPC A61B 5/165; A61B 5/18; A61B 5/486; A61B 5/742; G16H 10/20; G16H 40/67; A61B 2503/22

(57) ABSTRACT

A method for evaluating the fatigue level of an operator during a mission, the method including the following phases implemented by one or more portable evaluation devices: an initial collection phase implemented before the mission and including an operation of acquiring personal and physiological data of the operator, and a final collection phase implemented after the mission and including an operation of acquiring data related to the mission, the method further including an analysis phase including an operation of analyzing all the collected data and an operation of determining the operator's fatigue level before and/or after their mission through this analysis.

### CROSS-REFERENCE TO RELATED APPLICATION

This application is a U.S. non-provisional application claiming the benefit of French Application No. 24 03800, filed on Apr. 12, 2024, which is incorporated herein by reference in its entirety.

### TECHNICAL FIELD OF THE INVENTION

The present invention relates to a method for evaluating the fatigue level of an operator.

The present invention also relates to a system for evaluating the fatigue level that allows the implementation of such an evaluation method.

The invention is in the technical field of evaluating the fatigue of an operator who must perform a mission. The operator may be aircrew personnel in the aeronautical field or any other field where managing operator fatigue is a significant issue. These fields notably include those where operational continuity of the operator is necessary throughout their mission.

The invention particularly allows optimizing risk management related to operators in a critical field such as the aeronautical field concerning their actual fatigue state.

### BACKGROUND OF THE INVENTION

According to the state of the art, operator fatigue is generally analyzed during temporary campaigns based on questionnaires capturing subjective fatigue or based on individual fatigue declarations.

These two modes of capturing fatigue only allow subjective fatigue to be deduced, which may be biased by cultural or company pressure. In particular, it has been observed that operators tend to underestimate their fatigue.

The use of biomathematical models to predict the fatigue level of operators is also known.

However, these models generally use average data or declarative data from operators.

Existing solutions are unsatisfactory as they mainly rely on declarative and/or average data and therefore do not allow for an objective and reliable evaluation of the operators' fatigue level.

### SUMMARY OF THE INVENTION

The aim of the present invention is to propose a technique for evaluating the fatigue level of operators that provides objective and reliable results.

To this end, the invention aims at a method for evaluating the fatigue level of an operator during a mission.

The method includes the following phases implemented by one or more portable evaluation devices:
an initial collection phase implemented before the mission and including an operation of acquiring personal and physiological data of the operator;
a final collection phase implemented after the mission and including an operation of acquiring data related to the mission.

The method further includes an analysis phase including an operation of analyzing all collected data and an operation of determining the operator's fatigue level before and/or after their mission through this analysis.

The invention thus allows the fatigue level of an operator to be evaluated based not only on subjective data but also on objective data such as the operator's physiological data.

Furthermore, the invention proposes collecting these different types of data before and after the mission, which allows the impact of the mission on the operator's fatigue level to be estimated.

The invention also allows the evolution of fatigue induced by the tasks accomplished to be analyzed by offering an objective means of evaluating fatigue before and after the mission.

The fatigue level of operators determined by the invention may thus be used to identify situations conducive to the emergence of fatigue and then to manage or avoid said situations. This ensures the safety of the mission performed by the operators.

According to particular embodiments of the invention, the method includes one or more of the following features, taken individually or in any technically possible combination:
an intermediate collection phase implemented during the mission and including an operation of acquiring data related to the operator and/or the mission by a mobile device other than the portable evaluation device and/or by an onboard device;
the final collection phase further includes an operation of retrieving data acquired during the intermediate collection phase by transferring this data from the mobile device or the onboard device to the corresponding portable evaluation device;
the initial collection phase further includes an operation of acquiring at least one type of data chosen from the group including:
   subjective evaluation of fatigue by the operator;
   data on the planning of past missions;
   data on the nature and difficulty of past missions;
   data on the operator's sleep and naps;
   data on the operator's past activities;
   data on the mission to be performed;
the final collection phase further includes an operation of acquiring at least one type of data chosen from the group including:
   subjective evaluation of fatigue by the operator;
   mission characteristics data;
   subjective evaluation of the mission's difficulty;
the intermediate collection phase further includes an operation of acquiring at least one type of data chosen from the group including:
   subjective evaluation of fatigue by the operator;
   mission progress;
   significant events of the mission;
the analysis phase further includes an operation of displaying a recommendation to the operator determined based on their fatigue level;
the initial collection phase and the final collection phase are implemented by different portable evaluation devices communicating with each other either directly or via a remote server or through a mobile device used during an intermediate collection phase;
when the analysis phase is implemented after the final collection phase, the analysis operation includes a comparison of the data collected during the initial collection phase and during the final collection phase;
when the analysis phase is implemented at least once following the initial collection phase and at least once following the final collection phase.

The invention also aims at a system for evaluating the fatigue level of an operator, including means configured to implement the method as defined above.

### BRIEF DESCRIPTION OF THE DRAWINGS

The invention and its advantages will become apparent from the following description, given solely by way of non-limiting example and made with reference to the appended drawings wherein:

FIG. 1 is a schematic representation of a system for evaluating the fatigue level of an operator according to the invention;

FIG. 2 is a schematic view of a portable evaluation device that is part of the evaluation system of FIG. 1;

FIG. 3 is a flowchart of an evaluation method according to the invention, the method being implemented by the evaluation system of FIG. 1; and

FIGS. 4-6 are different illustrations of the implementation of the evaluation method of FIG. 3.

### DETAILED DESCRIPTION

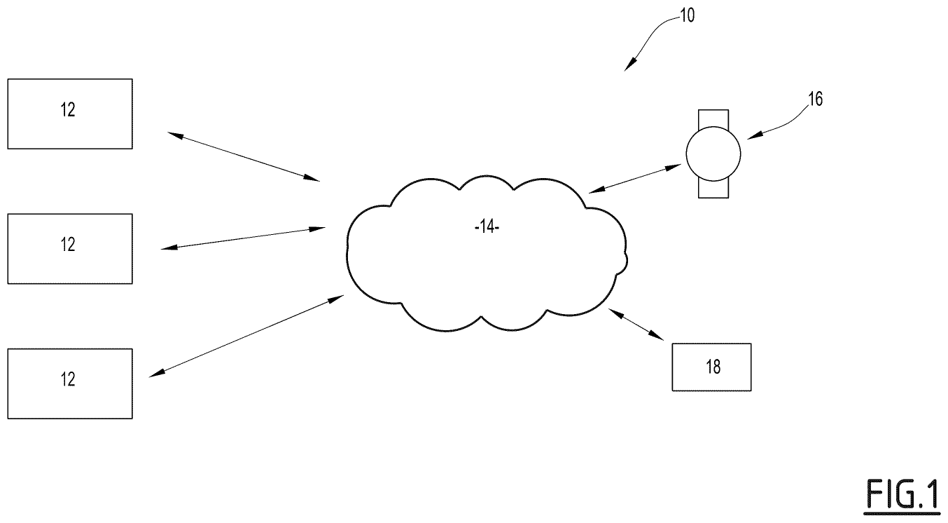

Indeed, FIG. 1 shows an evaluation system 10 for the fatigue level of an operator.

Advantageously, the evaluation system 10 is usable in the aeronautical field. In such a case, the operator is part of the aircrew, notably commercial aircrew. According to other examples, the operator is part of flight planning operators or maintenance operators or aircraft control operators or air traffic controllers.

Advantageously, the operator is a pilot capable of piloting an aircraft.

By aircraft, we mean any flying machine that may be piloted from its cockpit, as is the case, e.g., with an airplane or a helicopter, or remotely from it, as is the case, e.g., with a drone.

In general, the notion of the operator may apply to any other person performing a critical mission, e.g., in the transport field (rail or heavy goods, e.g.) or in the nuclear or space field, or in medicine.

As hereinabove indicated, the operator performs a mission determined by the field of their activity.

In particular, the operator's mission includes a plurality of tasks defined according to the operator's skills.

When the operator is an aircraft pilot, their mission generally consists of piloting the aircraft from a starting point to a destination point.

The evaluation system 10 according to the invention allows determining the operator's fatigue level.

To do this, the evaluation system 10 includes at least one portable evaluation device 12.

Advantageously, the evaluation system includes several portable evaluation devices 12 connected to each other in order to transmit computer data.

Optionally, and as illustrated in FIG. 1, the evaluation system 10 includes a remote server 14 allowing data to be exchanged between the different portable evaluation devices 12. The remote server 14 also allows computer data from at least some portable evaluation devices 12 to be stored and, in some cases, this data to be processed.

In some embodiments, the evaluation system 10 further includes a mobile device 16 that has a different structure from each of the portable evaluation devices 12, as will be explained in more detail later.

Also, in some embodiments, the evaluation system 10 further includes a plurality of sensors 18 installed, e.g., in the operator's workstation, such as the cockpit when said operator is an aircraft pilot.

The mobile device 16 and possibly the sensors 18 are also connected to the portable evaluation devices 12 directly or indirectly, e.g., via the same server 14 as illustrated in the main figure.

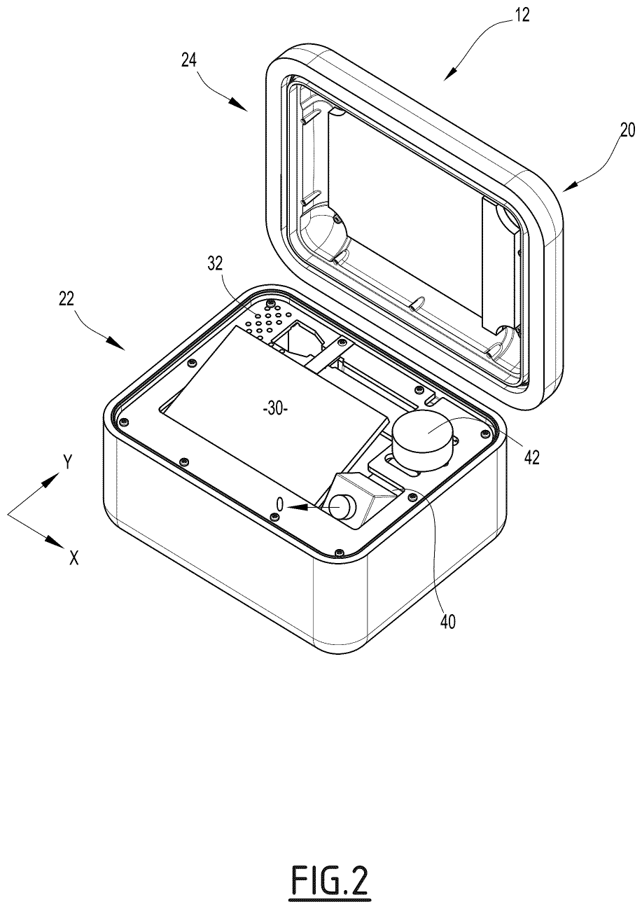

FIG. 2 illustrates in more detail a possible embodiment of a portable evaluation device 12.

Thus, as shown in FIG. 2, the portable evaluation device 12 has a case 20 integrating different internal components of this portable evaluation device 12.

In particular, the case 20 is, e.g., in the form of a suitcase or any other object that may be easily transported. In the example of FIG. 2, the case 12 is composed of two half-shells 22, 24. The case 20 may also include any other device facilitating its transportation, such as a handle, wheels, etc.

At least one of the half-shells, e.g., the half-shell 24, then forms an opening of the case 20. This half-shell 24 is movable between a closed position and an open position. In the open position, illustrated in FIG. 2, the half-shell 24 then allows at least partial access to the internal components of the portable evaluation device 12.

In general, the case 20 includes a plurality of components accessible by the operator when the half-shell 24 is in its open position and a plurality of components inaccessible by the operator in any position of the half-shell 24.

Among the components accessible by the operator, the portable evaluation device 12 notably includes means of interaction with the operator and a plurality of sensors.

The means of interaction with the operator notably includes visual interaction means such as a screen 30 and auditory interaction means such as, e.g., a speaker 32. The screen 30 and the speaker 32 are, e.g., integrated into an inner surface of the half-shell 22, which is intended to be protected by the half-shell 24 when same is in the closed position thereof.

The plurality of sensors includes any sensor allowing the physiological data of the operator to be acquired.

In particular, in the example of FIG. 2, the plurality of sensors includes a camera 40 configured to acquire images of the operator and a sensor 42 allowing the operator's heart rate to be measured.

The camera 40 is advantageously oriented towards the operator or has means allowing said camera to be oriented according to the operator's position.

The heart rate sensor 42 of the operator is advantageously removable from the case 20, e.g., to be positioned around the operator's wrist.

To this end, the sensor 42 has, e.g., a bracelet that may be attached onto the operator's wrist and a sensitive part intended to measure the operator's heart rate when the bracelet is attached onto their wrist.

The heart rate measurement is carried out, e.g., by the sensitive part using the technique called photoplethysmography, known as PPG. Alternatively, the sensitive part is configured to perform the heart rate measurement from an analysis of the electrical response by the operator's wrist or by analyzing radar signals propagating in the operator's wrist.

In some examples, the sensor 42 is configured to measure other physiological parameters of the operator, such as blood pressure, oxygen intake, sweating, dehydration rate.

For oxygen saturation, the sensor 42 is, e.g., configured to emit towards the operator's skin and receive a light signal including at least two wavelengths. A first wavelength corresponds to a wavelength absorbed by saturated red blood cells. A second wavelength corresponds to a wavelength absorbed by unsaturated red blood cells. To determine oxygen saturation, the sensor 42 is then configured to compare the light intensity received in response to each of the two wavelengths.

In general, the sensor 42 may be in the form of a connected watch that may then be stored in the case 20 when necessary or worn by the operator to, e.g., measure their heart rate.

Of course, the aforementioned functionalities of the sensor 42 may form separate sensors that may then be arranged in any possible configuration inside the case 20 or on a surface thereof.

The non-accessible components of the case 20 are notably arranged in the inner part of the case 20, e.g., in the inner part of the half-shell 22, and notably include a calculator, a memory, and a power module.

The calculator notably includes a processor allowing a plurality of applications that are stored to be executed, e.g., in the memory of the case.

The calculator further includes means of communication with external devices, notably with other portable evaluation devices 12 or with the server 14.

The power module allows all the components of the portable evaluation device 12 to be powered. This module includes, e.g., a battery providing autonomous power to these components. This battery may be associated with a charging device allowing the battery to be connected to an electrical network for recharging.

Returning to the description of FIG. 1, the mobile device 16 has any device allowing data to be acquired and stored during the operator's mission and transmitting said data to one of the portable evaluation devices 12.

Alternatively, the mobile device is only configured to store the data acquired during the initial collection phase P1. This storage advantageously allows this data to be sent from one point to another during the operator's mission. This data is then transmitted to one of the portable evaluation devices 12.

Advantageously, the mobile device 16 also allows physiological data of the operator to be acquired. To this end, the mobile device 16 includes, e.g., a sensor similar to the sensor 42 described hereinabove.

Such a sensor allows, e.g., the operator's heart rate or other physiological data of the operator to be measured as described hereinabove.

The mobile device 16 advantageously is a connected watch.

In some embodiments, the mobile device 16 and the sensor 42 as described hereinabove are the same entity. In such a case, the mobile device 16 is intended to be stored in the case 20 and worn by the operator during the operator's mission.

The sensors 18 present the same type of sensors as those described in relation to the portable evaluation device 12. Unlike the latter, said sensors are, e.g., installed in the operator's workstation. Furthermore, each of the sensors 18 allows the collected data to be sent to at least one of the portable evaluation devices 12 via, e.g., the mobile device 16 and/or the remote server 14.

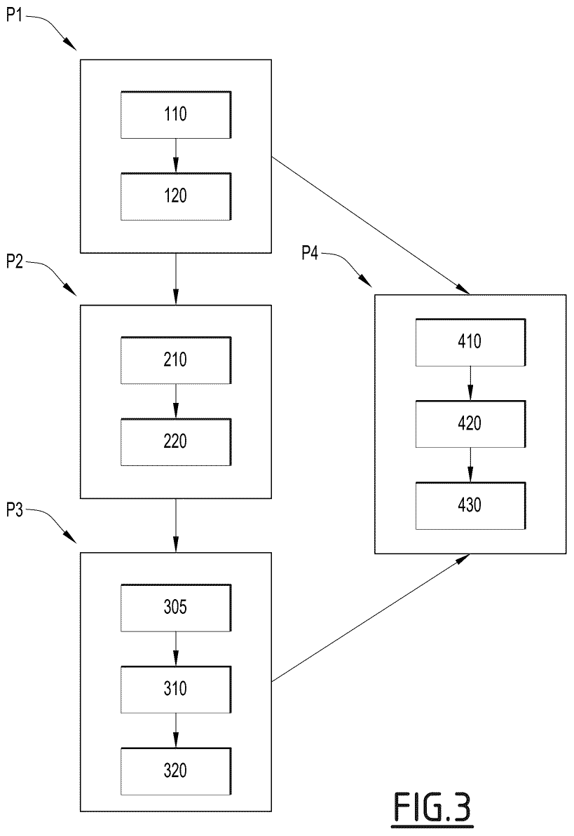

The evaluation system 10 allows a method to be implemented for evaluating the operator's fatigue level, said method now being described with reference to FIG. 3 presenting a flowchart of the operations thereof.

The method according to the invention notably includes an initial collection phase P1, which is implemented before the operator's mission.

In particular, this phase P1 is implemented by one of the portable evaluation devices 12.

To implement this phase P1, the corresponding portable evaluation device 12 is, e.g., placed in front of the operator, e.g., during the preparation of the upcoming mission.

Furthermore, when the portable evaluation device 12 is in the form of a suitcase, this device 12 is in an open position in front of the operator so that the sensors thereof are oriented towards the operator. The operator may also, e.g., put the sensor 42 on their wrist when said sensor is a removable sensor.

The portable evaluation device 12 then implements an operation of acquiring 110 personal and physiological data of the operator.

In particular, to acquire the operator's personal data, the device 12 interacts with the operator via the screen 30 and possibly via the camera 40. For example, the device 12 displays on the screen 30 a series of questions that the operator must answer to enter their personal data.

The operator's personal data includes, e.g., their identifier or their first and last name, an age range, etc.

According to some examples, only the operator's identifier is collected during this operation. This identifier may, e.g., be anonymized.

The operator's physiological data includes, e.g., all the data acquired by the plurality of sensors of the device 12. Thus, e.g., this data may include the operator's heart rate acquired by the sensor 40.

The operator's physiological data may also include images of the operator acquired by the camera 40 over a given interval. This interval may extend, e.g., up to a few minutes and may be implemented when the operator enters their data using the screen 30 or when they prepare their mission, e.g., by discussing this mission with other operators or managers.

The initial collection phase P1 further includes an operation of acquiring 120 additional data related to the operator and/or the upcoming mission or past missions.

For example, concerning the operator, the data acquired during this operation may include data on the sleep or nap taken by the operator. This data may include, e.g., the start time of sleep and end time, sleep quality, etc. thereof.

The data related to the operator may also include data related to activities performed by the operator in the days preceding the mission.

These activities may include in-flight and on-ground activities, training, on-call duty, illnesses, etc.

Finally, the data related to the operator may also include a subjective estimation of their perceived fatigue level or optionally their stress level or mental load, etc. To enter this data, the device 12 may, e.g., propose that the operator estimate their fatigue level on a given scale, their stress level, or their mental load on scales adapted for this purpose.

The information related to the mission to be performed may include, e.g., the position to be occupied during this mission (pilot, co-pilot, or other critical position, e.g., in nuclear or air traffic control), team composition (e.g., the number of pilots needed for the flight to be performed), the position of the workday in the period (i.e., the day number in the work sequence), information on mission planning (planned mission, re-planned mission, and in this latter case, the history of this re-planning), mission time range (morning, daytime, evening, nighttime), type of aircraft or any other workstation on which the mission will be performed.

The data related to past missions may include, e.g., the same data as for the mission to be performed. This data may also include difficulties encountered during these missions.

According to some embodiments, the device 12 then implements an analysis phase P4 allowing the data collected during the initial collection phase P1 to be analyzed.

This analysis phase P4 notably include an operation of analyzing 410 all the data collected during the initial collection phase P1 and an operation of determining 420 the operator's fatigue level before the mission following the analysis of the data collected during the initial collection phase P1.

To do this, the device 12 analyzes the declarative data entered by the operator but also the physiological data acquired by the sensors of the device 12 as explained hereinabove.

For example, the analysis of the images acquired by the camera 40 may include detecting the operator's fatigue level based on their eye blink frequency and/or their yawning frequency or other facial gestures.

The physiological data may then be superimposed with the declarative data to determine the operator's fatigue level, accurately and objectively.

This fatigue level may then represent a value chosen on a scale, e.g., on a numerical scale.

The fatigue level determined during this operation 420 may be displayed to the operator via the screen 30 or may be transmitted to their superior or stored on an external server (e.g., on the remote server 14) securely.

When the fatigue level display is performed on the screen 30, this display may take various forms chosen based on this level.

In some embodiments, the analysis phase P4 may also include a recommendation operation 430 implemented based on the fatigue level determined during operation 420.

For example, when the fatigue level is considered too high, the recommendation given by the device 12 may consist of postponing the mission or taking necessary measures during the mission to perform it safely.

In some embodiments, the analysis phase P4 may also include an operation of transmitting all the collected data to the remote server 14.

In some embodiments, the analysis phase P4 is not implemented immediately after the initial collection phase P1 but only after the final collection phase P3, as will be explained later. In such a case, the data collected during this initial phase may be transmitted to the remote server 14 or the mobile device 16.

The next phase, called the intermediate collection phase P2, is optionally implemented after the initial collection phase P1. In particular, this intermediate collection phase P2 is implemented during the operator's mission by the mobile device 16 as explained hereinabove.

Alternatively or in addition, this intermediate collection phase P2 is also implemented by the sensors 18, as explained hereinabove.

In particular, this intermediate collection phase P2 may include an operation of acquiring 210 data related to the operator and/or the mission by the mobile device 16.

The data related to the operator may include, e.g., physiological measurements of the operator, such as their heart rate, e.g., throughout this mission.

The data related to the mission may include, e.g., information on the mission's progress, such as its start, duration, end, etc.

The intermediate collection phase P2 may also include an operation 220 of acquiring additional data.

This additional data may be related to the mission's progress (takeoff, end of climb, start of descent, landing) when the operator is an aircraft pilot, as well as significant events of the mission (failure, turbulence, weather, crew disturbance).

The additional data may also include subjective estimates of the perceived fatigue state and optionally the operator's stress level or mental load.

The operator may, e.g., enter these estimates several times during their mission. These estimates may be timestamped.

The method according to the invention further includes a final collection phase P3, which is implemented after the mission. This final collection phase P3 may then be implemented following the intermediate collection phase P2 when it has been implemented during the operator's mission or directly after the initial collection phase P1.

This final collection phase P3 is implemented, just like the initial collection phase P1, by a portable evaluation device 12. This device may, e.g., be the same as the device 12 used to implement the initial collection phase P1 or another similar device.

The final collection phase P3 may include an operation 305 of acquiring the data acquired during the intermediate collection phase P2 when this latter phase has been implemented during the operator's mission.

During this operation 305, the data acquired by the mobile device 16 or the sensors 18 are transferred to the portable evaluation device 12. This may be done, e.g., by connecting this device 16 directly to the device 12. When it comes to the sensors 18 installed, e.g., in the operator's workstation, the acquired data may be transmitted using an external server (e.g., the remote server 14).

The final collection phase P3 may also include an operation of acquiring 310 data related to the mission performed by the operator. This data related to the mission may include, e.g., the type of mission (training, operational, evaluation, transit), mission duration, activities during the mission (piloting activity, radio and other systems management), the presence of rest during the mission and the duration of this rest, levels of disturbance during the mission, workload during the mission (low, medium, high).

The final collection phase P3 may also include an operation of acquiring 320 additional data.

This additional data may include, e.g., a subjective evaluation of fatigue by the operator and a subjective evaluation of the mission's difficulty.

These evaluations may be entered by the operator on a determined scale.

The additional data may also include other characteristic data of the mission not hereinabove mentioned.

These characteristic data may correspond, e.g., to technical data related to the mission's progress.

The portable evaluation device 12 then implements the analysis phase P4 as explained hereinabove.

In particular, unlike the previous explanations, operation 410 of this phase includes the analysis of all the data collected during the previous phases.

To do this, the portable evaluation device 12 may access the data collected during the initial collection phase P1, e.g., on the remote server 14 or via the mobile device 16. Thus, the analysis operation 410 includes the analysis of all the data collected during all the previous collection phases. This analysis operation 410 may also include a comparison of the data collected during the initial collection phase P1, and during the final collection phase P3 and possibly during the intermediate collection phase P2.

Then, operation 420 determines the operator's fatigue level using all the collected and analyzed data.

As in the previous case, the operator's fatigue level determined at this stage may be displayed to the operator via the screen 30 or transmitted to their superior.

Furthermore, as in the previous case, the analysis phase P4 may include the recommendation operation 430. In this case, the recommendation given to the operator takes into account that the mission has already been performed. The recommendation given at this operation may, e.g., consist of the recommended amount of rest hours for the operator.

As hereinabove indicated, the analysis phase P4 is implemented either by the device 12 implementing the initial collection phase P1 or by the device 12 implementing the final collection phase P3.

However, in a general case, this phase P4 may also be implemented independently of these devices, e.g., by an external calculator connected to said device 12, e.g., via the server 14 to collect the acquired data.

The server 14 may also keep all the collected and analyzed data for future use of this data, e.g., to determine a corresponding statistic.

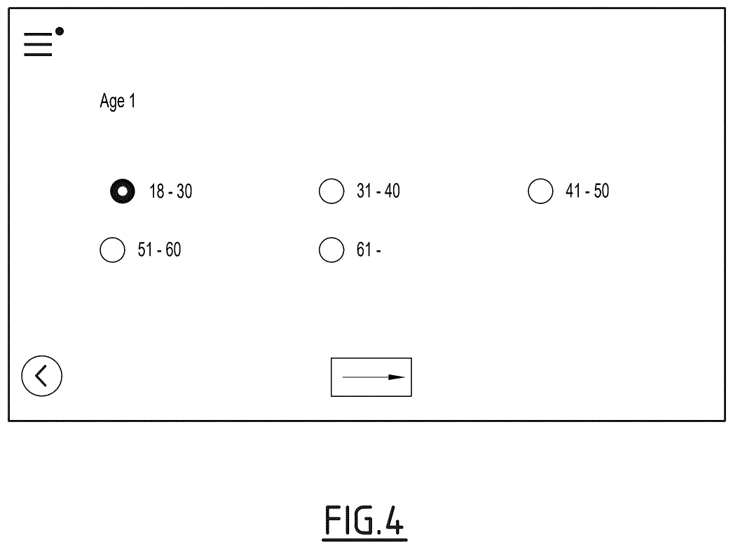

FIGS. 4 to 6 illustrate an implementation of certain operations described hereinabove.

Thus, e.g., FIG. 4 illustrates operation 110 of acquiring the operator's personal data. During this operation, the device 12 notably proposes that the operator enter their age range as illustrated in FIG. 4.

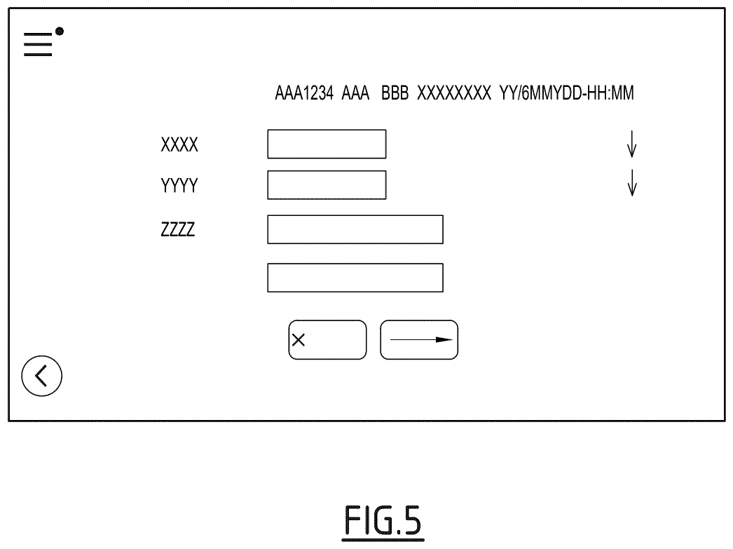

FIG. 5 illustrates an example of the implementation of operation 310 of acquiring data related to the mission.

For example, during this operation, the device 12 invites the operator to enter weather data related to the mission. For this, the system 12 proposes, e.g., different choices among possible options.

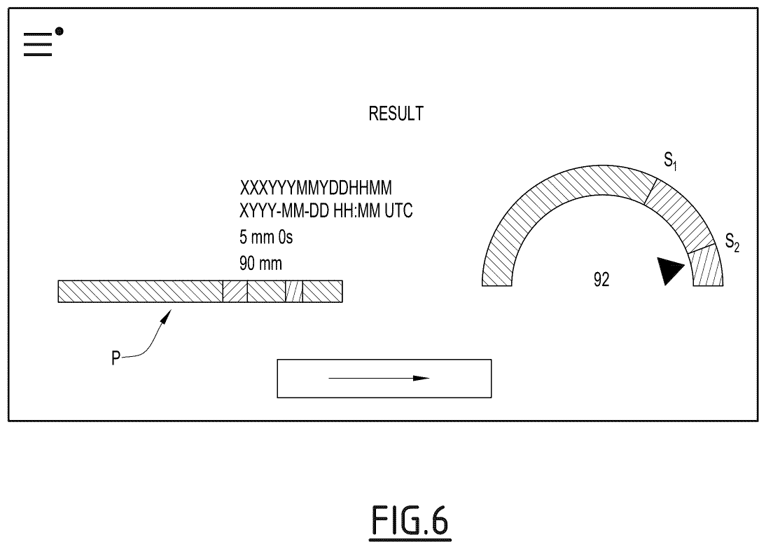

Finally, FIG. 6 illustrates an implementation of operation 420 during which the fatigue level determined by the analysis phase P4 is displayed to the operator.

As illustrated in this figure, the assigned fatigue level may be assigned, e.g., on a scale from 0 to 100. This scale also defines some thresholds allowing a quick evaluation of the fatigue level. For example, in this figure, two thresholds S1 and S2 are determined.

When the fatigue level is below threshold S1, the fatigue level is considered normal. When the fatigue level is between the two thresholds S1 and S2, this level is considered high. When the fatigue level is above threshold S2, it is considered very high.

FIG. 6 also illustrates a temporary scale P of fatigue level evolution, e.g., during the mission.

Of course, many other illustrations of the implementation of the aforementioned operations are also possible.

Alternatively or in addition, when the operator is determined to be fatigued during the analysis phase P4, i.e., their fatigue is above threshold S1, their fatigue is categorized. Their fatigue then belongs to a specific type determined by causes related to their mission. For example, an abnormally long mission will induce fatigue categorized as long-term or chronic fatigue. In contrast, intense effort such as a mission with many unforeseen incidents will induce fatigue of the high mental load type or acute fatigue.

Other causes of fatigue are related to events outside their mission. For example, the operator's fatigue may be categorized as emotional following a personal event. The operator's fatigue may also be related to medical conditions or lack of sleep.

The operator's fatigue may also be classified with several types of fatigue when it fulfills several causes.

Advantageously, this variant allows the operator's fatigue to be contextualized.

1. An evaluation method of the fatigue level of an operator during a mission, the method comprising the following phases implemented by one or more portable evaluation devices:
an initial collection phase implemented before the mission and comprising acquiring personal and physiological data of the operator;
a final collection phase implemented after the mission and comprising acquiring data related to the mission; and
an analysis phase comprising:
analyzing all the acquired data; and
determining the operator's fatigue level before or after their mission through this analysis.

2. The method according to claim 1, wherein said determining occurs before and after the mission through said analyzing all the collected data.

3. The method according to claim 1, further comprising an intermediate collection phase implemented during the mission and comprising acquiring data related to the operator and/or the mission by a mobile device other than the portable evaluation device and/or by an onboard device.

4. The method according to claim 3, wherein said intermediate collection phase further comprises acquiring at least one type of data chosen from the group comprising:
subjective evaluation of fatigue by the operator;
mission progress; and
significant events of the mission.

5. The method according to claim 3, wherein said final collection phase further comprises retrieving the data acquired during said intermediate collection phase by transferring this data from the mobile device or the onboard device to the corresponding portable evaluation device.

6. The method according to claim 1, wherein said initial collection phase further comprises acquiring at least one type of data chosen from the group comprising:
subjective evaluation of fatigue by the operator;
data on the planning of past missions;
data on the nature and difficulty of past missions;
data on the operator's sleep and naps;
data on the operator's past activities; and
data on the mission to be performed.

7. The method according to claim 1, wherein said final collection phase further comprises acquiring at least one type of data chosen from the group comprising:
subjective evaluation of fatigue by the operator;
mission characteristics data; and
subjective evaluation of the mission's difficulty.

8. The method according to claim 1, wherein said analysis phase further comprises displaying a recommendation to the operator determined based on their fatigue level.

9. The method according to claim 1, wherein said initial collection phase and said final collection phase are implemented by different portable evaluation devices communicating with each other either directly or via a remote server or through a mobile device used during an intermediate collection phase.

10. The method according to claim 1, wherein when said analysis phase is implemented after said final collection phase, and wherein said analyzing comprises comparing data collected during said initial collection phase and during said final collection phase.

11. The method according to claim 1, wherein said analysis phase is implemented at least once following said initial collection phase and at least once following said final collection phase.

12. The method according to claim 1, wherein a mobile device other than the portable evaluation device is configured to store, during the operator's mission, the data acquired during said initial collection phase to transmit the data to one of the portable evaluation devices.

13. The method according to claim 1, wherein, when the operator is determined to be fatigued during said analysis phase, their fatigue belongs to a specific type determined by causes whether or not related to their mission.

14. An evaluation system of the fatigue level of an operator, comprising calculating modules configured to implement the method according to claim 1.
